(19) United States
(12) Patent Application Publication
(10) Pub. No.: US 2008/0269767 A1
(43) Pub. Date: Oct. 30, 2008

(54) INTRA-OPERATIVE T-SQUARE

(76) Inventor: Michael F. O'Brien, Pinecrest, FL (US)

Correspondence Address: PAUL D. BIANCO;Fleit Gibbons Gutman Bongini & Bianco PL, 21355 EAST DIXIE HIGHWAY, SUITE 115, MIAMI, FL, 33180, US

(21) Appl. No.: 12/062,229

(22) Filed: Apr. 3, 2008

Related U.S. Application Data
(60) Provisional application No. 60/909,720, filed on Apr. 3, 2007.

(51) Int. Cl. A61B 17/58

(52) U.S. Cl. 606/102

(57) ABSTRACT

A T-square apparatus that facilitates intra-operative applications of orthogonal alignment to spinal reconstruction. More specifically, at least one cross member is connected orthogonally to a longitudinal member to assist in aligning the hips and pelvis perpendicular to the longitudinal access of the spine and to assist in positioning of the shoulders parallel to the hips and perpendicular to the spine. The T-square apparatus includes radiopaque markers to allow more accurate estimation of this sometimes difficult to appreciate intra-operative alignment.

### CROSS REFERENCE TO RELATED APPLICATION

This application claims the benefit under 35 U.S.C. 119(e) of U.S. Provisional Patent Application No. 60/909,720, filed Apr. 3, 2007, the entirety of which is incorporated herein by reference.

### FIELD OF THE INVENTION

The present invention relates to a device for facilitating intra-operative applications of orthogonal alignment to spinal reconstruction. More specifically, the invention will assist in aligning the hips and pelvis in a position perpendicular to the longitudinal axis of the spine and assist in positioning the shoulders parallel to the hips and perpendicular to the spine.

### BACKGROUND OF THE INVENTION

Chronic back problems cause pain and disability for a large portion of the population. In many cases, the chronic back problems are caused by intervertebral disc disease and loss of stability of the intervertebral joint. However, stabilization and arthrodesis of the intervertebral joint can reduce the painful affects associated with chronic back problems.

Spinal fusion surgeries were developed many years ago to stop the motion at a painful vertebral segment, which in turn should decrease pain generated from the joint. Most fusion surgery techniques involve removing some or all of the diseased disc material and adding bone graft to an area of the spine to set up a biological response that causes the bone graft to grow between the two vertebral elements and thereby stop the motion at that segment.

The recent trend in spine surgery has moved toward minimally invasive procedures. Instead of performing open procedures requiring larger incisions, muscle stripping, more anesthesia, longer operating time, and longer hospitalization, minimally invasive surgery utilizes tiny incisions in which small specialized instruments and implants are inserted. Various equipment and devices are available to magnify and view these small areas.

A common problem in either spinal fusion surgery or minimally invasive procedures is that during the surgery, the inserting of a prosthesis and adjusting or removing bone tissue can result in misalignment of the spine or other anatomical parts of the patient. Misalignment of the hips, pelvis, spine, or shoulders can have serious adverse complications after surgery such as increased curvature of the spine and hips being unequal, with one higher than the other. These complications result in an increase of wear and tear on various joints of the patient causing significant pain. If the alignment is not fixed during surgery, another surgery may be required.

Misalignment of the spine often results in long term pain, uneven gait, osteoarthritis, and difficulty in performing functions of daily living. A mal-alignment is often difficult to assess and measure during the surgery. However, surgery is the crucial period because during surgery is when alignment can be corrected. Thus, a device is needed to facilitate orthogonal alignment during spinal reconstruction, and which can be used intra-operatively to assist in measurement of the position of the anatomical components of the spine and other anatomic structures prior to closing the wound when actions can be taken to correct a less than optimal measurement.

### SUMMARY OF THE INVENTION

The present invention is used to help facilitate intra-operative applications of orthogonal alignment to spinal reconstruction. The device will assist in aligning the hips and pelvis perpendicular to the longitudinal access of the spine and assist in positioning of the shoulders parallel to the hips and perpendicular to the spine. This anatomical positioning will help create appropriate coronal and sagittal balance postoperatively. The device could also be used to help align the hips in relation to the spine, independent of the ilium. It is a goal of the invention to allow more accurate estimation of the sometimes difficult to appreciate intra-operative alignment process during spinal surgery.

In another aspect of the invention, this device could be used in less rigorous spinal reconstructions to be sure that segments of the spine being fused, although not connected directly to the sacrum or the pelvis, are also aligned and orthogonal to the foundation of the sacrum, pelvis, and hip joints.

In accordance with an embodiment of the invention, a T-square shaped device is provided. The T-square device includes a longitudinal member and at least one cross member. The longitudinal member is a rod that runs along the length of the patient's spine, and the rod is used as the longitudinal visual marker for the spine. The cross member is a rod that is positionable orthogonally to the longitudinal member, and it serves as a visual marker for the hips, pelvis, shoulders or some other anatomic or extra-anatomic reference. More than one cross member could be connected to the longitudinal member so that the surgeon could check the hips, spinal alignment, and/or shoulder levels at the same time.

In an alternate embodiment of the present invention, the cross member could be connected to the longitudinal member so that it is operable to slide along the length of the longitudinal member. In this embodiment, one cross member can serve as the visual marker for multiple anatomic references by sliding the cross member along the longitudinal member in accordance with the anatomic reference that the surgeon wishes to check. The cross member could slide on a sliding dovetail or other sliding mechanism design.

In accordance with a further aspect of the present invention, the longitudinal member and/or cross members can be embedded with radiopaque wires or metallic markers to aid in the alignment process and to estimate various anatomic dimensions if the device is used during fluoroscopy.

In accordance with an alternate embodiment of the present invention, two T-square devices could be joined along their respective longitudinal members. In this embodiment, the cross member of the first T-square device is used as a reference line for shoulder alignment, the cross member of the second T-square device is used as a reference line for hip or pelvic alignment, and the joined longitudinal member is used as a reference line for the spine. In a preferred embodiment, the cross members are operable to slide along their respective longitudinal members and the longitudinal members of the two T-square device are also slideably engaged.

In accordance with a method of using the present invention, an anatomical reference is chosen. The anatomical reference is usually an anatomical reference line orthogonal to the spine, and the reference line intersects the spine at an anatomical intersection. The longitudinal member and cross member of the T-square device intersect at a device intersection. The device is positioned, with regard to the spine, by overlying the device intersection and the anatomical intersection. Following positioning of the device with respect to the spine, at least one of the longitudinal member and cross member is aligned with the spine and the anatomical reference line, respectively. The alignment of the longitudinal member with the spine and/or alignment of the cross member with the anatomical reference line can then be compared to assure proper alignment of the spine during surgery.

The subsequent description will elucidate several different versions of the T-square design, with various modifications in shape, material and manufacturing. They include but are not exclusive to the representative drawings. It is conceivable that this device could be made of various radiopaque and/or radiolucent materials, both metal, plastic and composite. The device also can be used for aligning occiput to cervical spine, cervical spine to the pelvis, and facilitate alignment of other appendicular and axial anatomy. The device can be sterilized and used during surgery or used nonsurgically. The device could be manufactured out of available stock material or molded, or machined from a variety of products.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a diagrammatic top-side view of a T-square device in accordance with the present invention;

FIG. 2 is a top perspective view depicting a cross member having a radiopaque wire and a plurality of holes;

FIG. 3 is a topside perspective view depicting a second embodiment of the T-square device having a telescoping rod disposed within a longitudinal member in accordance with the invention;

FIG. 4 is a topside view depicting a third embodiment of the T-square device having two cross members welded to an extendable telescoping longitudinal member in accordance with the invention;

FIG. 5 is a topside view depicting a fourth embodiment of the T-square device having a sliding cross member;

FIG. 5A is a front-side exploded view depicting the sliding dovetail connection for the cross member of the T-square device of FIG. 5;

FIG. 5B depicts an exploded view of the sliding dovetail connection for the radiolucent circular markers of the cross member in the T-square device of FIG. 5;

FIG. 6 is a front side detailed view depicting the cross member of the T-square device of FIG. 5;

FIG. 7 is an elevational view depicting the positioning of the cross members of a T-square device according to the present invention;

FIG. 8 is a side view depicting the T-square device in the position shown in FIG. 7;

FIG. 9 is a topside view depicting an alternate embodiment the device having two connected fixed T-pieces with cross members molded to their respective longitudinal members;

FIG. 10 is a perspective view depicting the T-square device of the present invention suspended above an operating table;

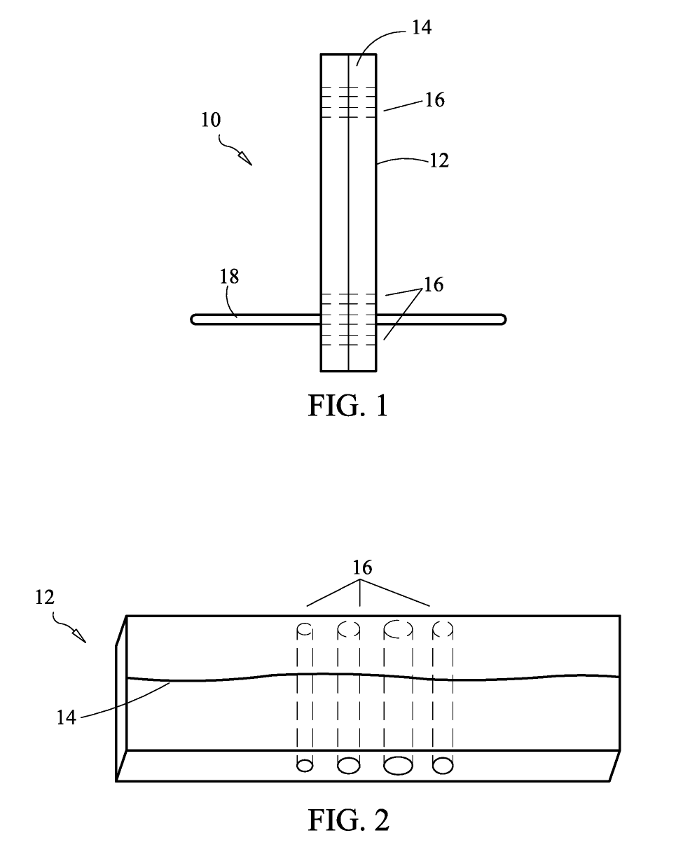

FIG. 1A-10B shows various possibilities for suspending the T-square device of the present invention using solid movable blocks.

### DETAILED DESCRIPTION OF THE INVENTION

The present invention is directed to a device for facilitating intra-operative applications of orthogonal alignment to spinal reconstruction. More specifically, the present invention is a T-square device that includes a longitudinal member and at least one cross member connected orthogonally to the longitudinal member. The device is to be positioned above the patient's back so that the longitudinal member is aligned with respect to the patient's spine and the cross members are aligned with respect to various anatomical references of the patient.

Referring now to the figures in which like reference numerals refer to like elements, an exemplary T-square device 10 according to the present invention is shown in FIG. 1. The longitudinal member 12 is a rectangular block having a radiopaque wire 14 or metallic marker disposed within it. The longitudinal member 12 could be either radiolucent or radiopaque. The radiopaque wire 14 is placed in the longitudinal member 12 either during molding of the part, placed into the part after machining an appropriate groove or space in the longitudinal member 12, or using some other implantation technique. The longitudinal member 12 further comprises at least one hole 16 for inserting a cross member 18. The holes 16 in the block should be drilled in an orthogonal orientation to the radiopaque wire 14. In a preferred embodiment, the holes 16 will have various diameters to accommodate various dimensioned cross members 18 that will be used for the particular patient or particular surgery.

In accordance with the embodiment of the invention shown in FIG. 1, the longitudinal member 12 and radiopaque wire 14 would serve as the longitudinal visual marker for the patient's spine. The cross members 18 are inserted orthogonally to the longitudinal member 12. The cross members 18 serve as a visual reference line for the hips, pelvis, shoulders or some other anatomic or extra-anatomic reference. The desired reference line depends on the cross member 18 chosen by the surgeon and the particular hole 16 the cross member 18 is inserted into. For example, if a cross member 18 is inserted at the proximal end of the longitudinal member 12 as shown in FIG. 1, the cross member 18 could be used to reference the hip joints. If a cross member 18 is inserted at the distal end of the longitudinal member 12, the cross member 18 could be used as a reference line for the shoulders. Multiple cross members 18 can be inserted into the longitudinal member 12 during surgery so that the surgeon could simultaneously check the hips, pelvis, spinal alignment, and/or shoulder levels of the patient.

Alternatively, the T-square device 10 described above could be turned around so that the longitudinal member 12 having the radiopaque wire 14 and plurality of holes 16 would now be a cross member (FIG. 2), and a longitudinal rod could be inserted into one of the plurality of holes 16 to be used as a reference line for the spine.

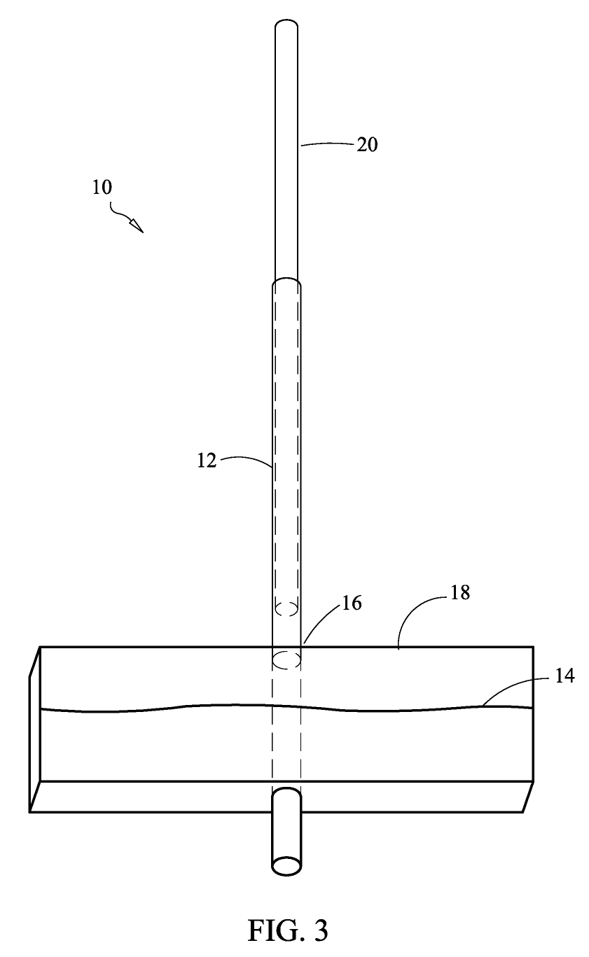

With reference to FIG. 3, a variation on the T-square device 10 having the cross member 18 as the rectangular block with a radiopaque wire 14 is shown. The longitudinal member 12 has a fixed diameter and is fitted through a hole 16 of the cross member 18. The longitudinal member 12 is positioned orthogonally to the cross member 18. The longitudinal member 12 of this device 10 includes an extendable telescoping rod 20 disposed within the longitudinal member 12. Preferably, the device 10 ought to be sized to be easily placed into an autoclave. Typically, an autoclave chamber has dimensions around 10 cm×40 cm×66 cm, and a large pan for an autoclave has dimensions around 10 cm×25 cm×51 cm. The telescoping rod device could be manufactured in various sizes as long as it was sufficiently rigid and had a small enough inner-outer diameter tolerance so that the extendable rod 20 did not have a lot of play in it.

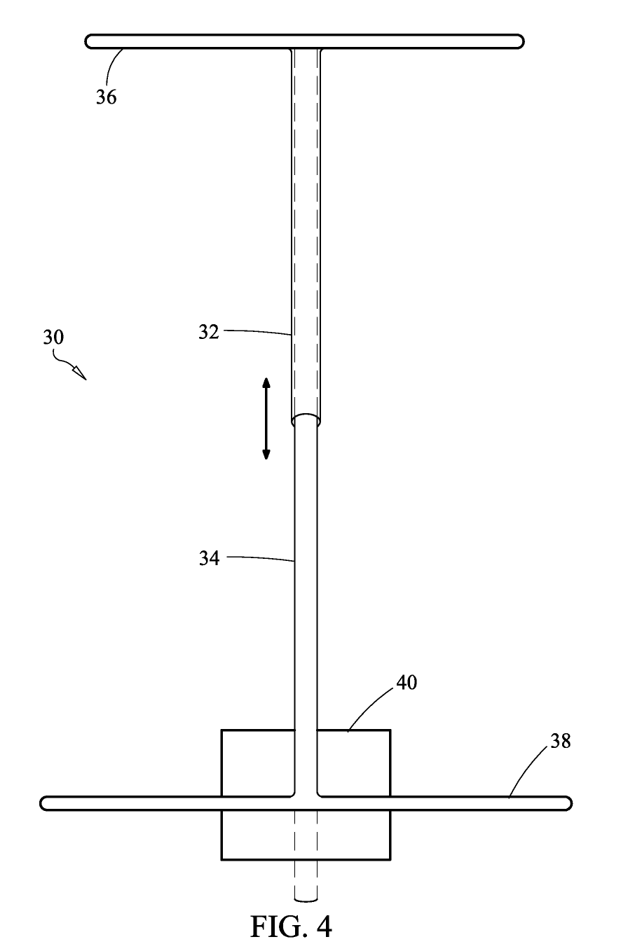

With reference to FIG. 4, and in accordance with another embodiment of the invention, the T-square device 30 has a first longitudinal member 32 and a second longitudinal member 34. The longitudinal members 32, 34 are preferably telescopic rods. The longitudinal members 32, 34 are operably connected to slide side by side or one can slide on top of the other. The device further includes two cross members 36, 38 welded at a ninety degree angle at the ends of their respective longitudinal member 32, 34. The distance between the cross members 36, 38 is adjusted by slideably extending the longitudinal members 32, 34. Because this configuration of the T-square device 30 may be susceptible to bending or weld failure, reinforcing of the cross members 36, 38 to the longitudinal members 36, 38 with a block of radioulucent material 40 is preferred.

Preferably, the upper cross member 36 is aligned with the patient's shoulders to allow for visualization of shoulder balance in relation to the patient's hips and spine. The lower cross member 38 is placed over the patient's hip joints or the ilium. However, alternate anatomical or extra-anatomical references could be used. Alternatively, the most simplistic form of this embodiment would have just a single cross member welded to one longitudinal member.

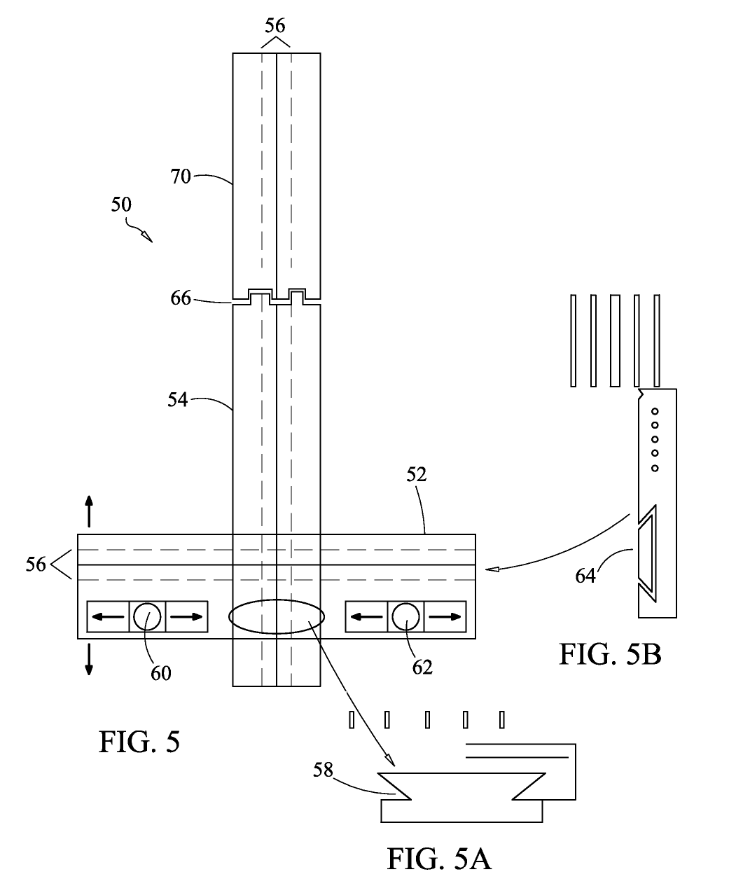

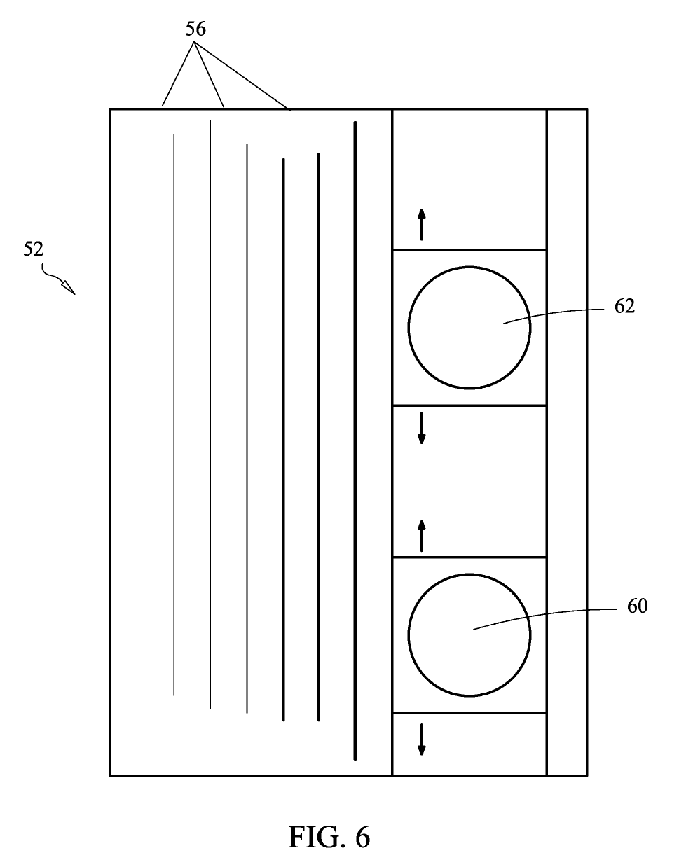

With reference to FIG. 5, in accordance with a further embodiment of the invention, the cross member 52 of the T-square device 50 is operable to slide along the longitudinal member 54. The longitudinal member 54 and cross member 52 are imbedded with several types of radiopaque markers/wires 56 (represented by dashed and solid lines) so that anatomic dimensions can be measured during fluoroscopy. Using the wires 56 to aid in measuring the anatomic dimensions helps quantify the amount of coronal and sagittal imbalance or other anatomic deflections or malalignments. The wires 56 should preferably be placed at fixed distances apart from each other as shown in the detailed view of the cross member 52 in FIG. 6. Furthermore, the wires 56 should preferably be of varying width, with the thickest wire centrally located.

As shown in FIG. 5, the cross member 52 of this embodiment is slidable from the distal end of the longitudinal member 54 when used as a shoulder reference line to the proximal end of the longitudinal member 54 when used as a hip or pelvic reference line. In a preferred embodiment, the cross member 52 slides using a sliding dovetail connection 58 as shown in the exploded view in FIG. 5A. However, other sliding mechanisms could be used. It may also be preferable to include a second cross member at the distal end of the longitudinal member 54. This cross member could also be capable of sliding along the longitudinal member 54.

The cross member 52 of this embodiment may also incorporate a first medial/lateral radiolucent circular marker 60 on one side of the cross member 52 and a second medial/lateral radiolucent circular marker 62 on the opposite side of the cross member 52. The first and second medial/lateral radiolucent markers 60, 62 are positionable over the femoral heads of the patient to ensure accurate localization of the T-square device 50. Positioning of the circular markers 60, 62 on their respective side of the cross member 52 may also be accomplished by using another sliding dovetail connection 64 (as shown in FIG. 5B) or other known sliding connections. FIG. 6 shows a detailed view of the cross member 52 and the radiolucent circular markers 60, 62 of the present embodiment.

Because the longitudinal member 54 of this embodiment of the present invention may be too long to fit into an autoclave, a hinge device 66 or non-hinged sliding connection may be needed to separate the longitudinal member 54 into two pieces 54, 70.

It is contemplated that the non-hinged sliding connections of the present invention could be either a datto, a sliding mortise/tenon, or some other sort of sliding dovetail connection. Other possible mechanical hinges could include a spring lock-loaded device, medial/lateral or rostral-caudal slides that will lock into place.

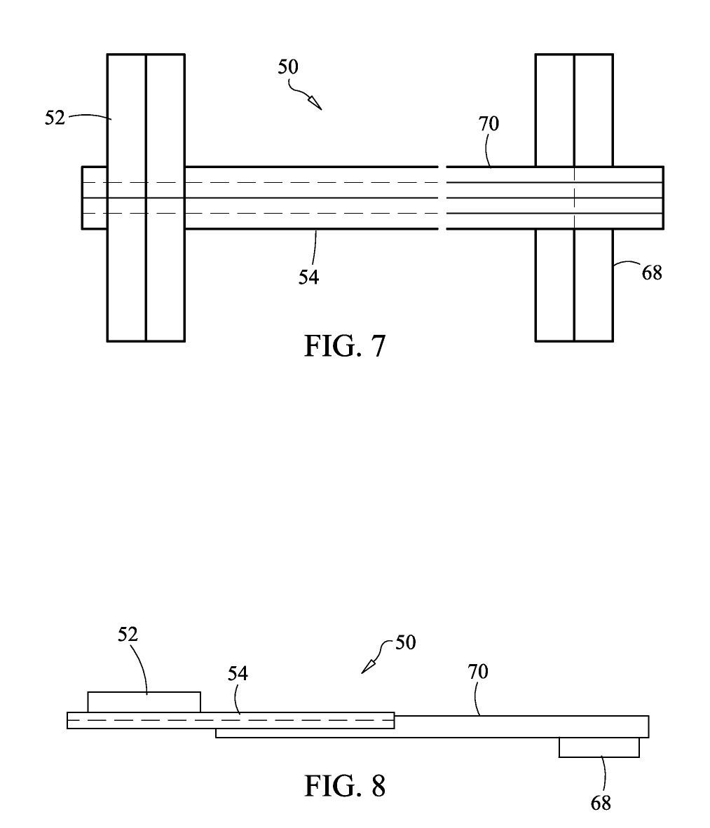

FIGS. 7 and 8 show two different views for possible positioning of the cross members 52, 68 of the T-square device 50 discussed with respect to FIG. 5. Two molded or machined pieces 54, 70 would serve as the longitudinal member. The pieces 54, 70 could be used independently or connected via one of the previously discussed connection options. Also, the two pieces 54, 70 of the longitudinal member may overlap as shown in FIG. 8, whereby a sliding dovetail is used to vary the length of the longitudinal member 54, 70. Each piece of the longitudinal member 54, 70 would have a cross member 52, 68 connected to either the top or bottom of its respective piece of the longitudinal member 54, 70. Furthermore, the cross members 52, 68 could be capable of sliding along the length of its respective piece of the longitudinal member 54, 70 using a dovetail or other rail. When using a sliding dovetail as the connection for the longitudinal member 54, 70, the width of the longitudinal member 54, 70 and the cross member 52, 64 should preferably be about 5 to 5 cm so that the sliding dovetail piece is wide enough to prevent deflection while the longitudinal member 54, 70 is extended and thick enough so that it will not break. There should also preferably be only one slide on each of the cross members 52, 68 so that there is enough of the dovetail engaged to maintain linear alignment of the slide.

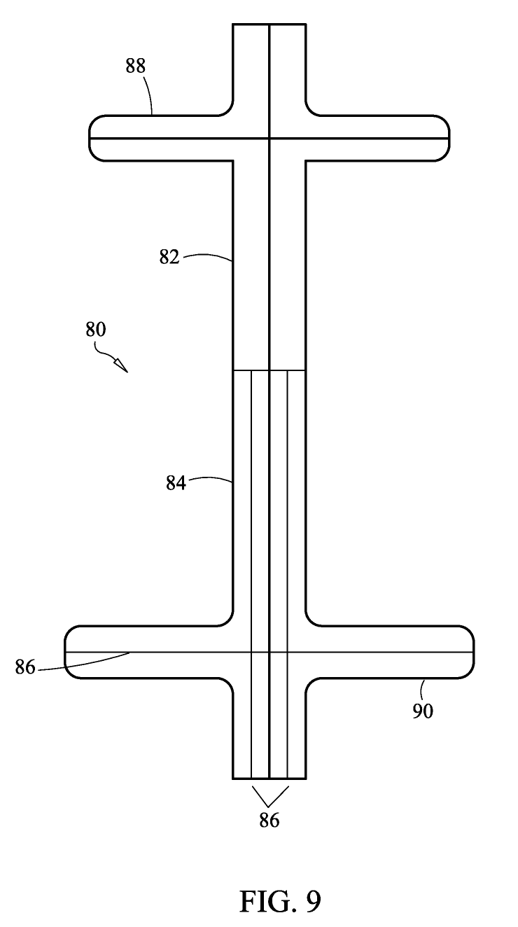

The embodiment of FIG. 9 shows a simplified version of the embodiment discussed above. This T-square device 80 consists of two fixed “T” shapes connected together along their longitudinal members 82, 84. This configuration would allow disassembly of either of the longitudinal members 82, 84 to save space and allow the two “T” pieces to be placed in an autoclave pan. Each of the “T” pieces is manufactured out of a single block. Radiopaque markers 86 may be embedded or molded into the radiolucent or radiopaque material of the longitudinal members 82, 84 and cross members 88, 90 or may be press-fit into the surface of the “T” piece via some milling design. The two pieces could either be used separately, or they could be connected via the sliding dovetail connection described above to allow the device 80 to be used over a longer or shorter linear distance. The sliding dovetail could be manufactured, either in part or completely, along the longitudinal members 82, 84 so that the two “T” pieces could overlap. The radiopaque markers 86 could run either above or below the female section of the sliding dovetail.

Alignment of the T-square device with respect to the patient can be accomplished in various ways. One way is having several pairs of superimposable radiopaque markers on the top and bottom of the cross members and the longitudinal member to orient the device within the fluoro field. This would help with orthogonal alignment of the T-square device and the patient to the x-ray beam using the technique of parallax. Alternatively, the orthogonal alignment can be achieved by other techniques such as a strip or piece of radiopaque material oriented in such a way that if it is not othrogonal to the x-ray beam, it will look differently than if it is orthogonal, i.e. a thin strip of metal cut into a rectangular shape would look like a line viewed on edge but would look like a rectangle when viewed enface. For hip alignment there can be sliding cross-hairs provided to locate the femoral head or the acetabulum.

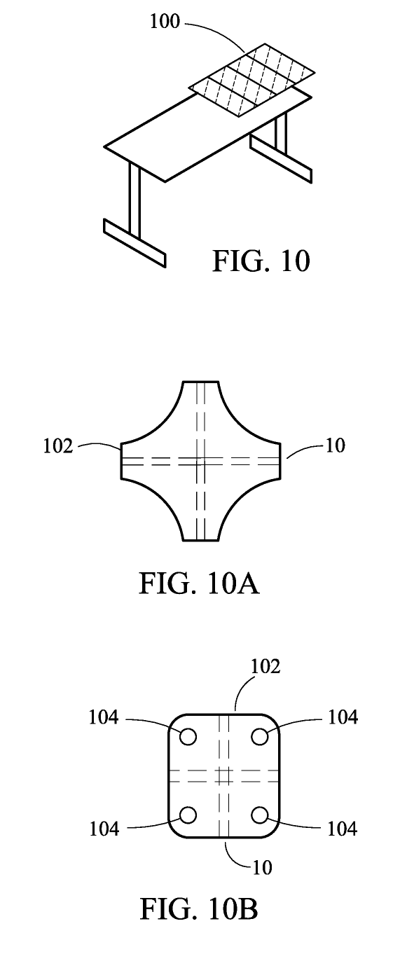

As shown in FIG. 10, a table mount 100 could be provided that holds the T-square device suspended above the patient during surgery or x-rays. FIGS. 10A and 10B show using a solid movable block 102 that can be mounted to an operating table as shown in FIG. 5 with orthogonal holes 104 drilled through to create the T-square. Leveling legs 104 are provided to level and raise the T-square off the patient to facilitate alignment to the patient. The leveling legs 104 could be made of either the same or different material as the radiolucent block. They could be solid posts, threaded screws, sliding posts, combination of the two, or some other adjustable or fixed configuration. The longitudinal member can be supported at the rostral end by either a small block with 1-2 posts, screws, or another large block can be placed to allow a cross member to help align the shoulders.

Alternatively, one may add a circular leveling bubble, two individual straight bubbles, or some other leveling device to level the T-square when it is suspended above the patient's back.

A method of using the device 10 for aligning the spine with respect to an anatomical reference is also encompassed by the invention. The anatomical reference is an anatomical reference line orthogonal to the spine, and the reference line intersects the spine at an anatomical intersection. The device 10 includes the longitudinal member 12 orthogonal to the cross member 18. The longitudinal member 12 and cross member 18 intersect at a device intersection. The device 10 is positioned, with regard to the spine, by overlying the device intersection and the anatomical intersection. Following positioning of the device with respect to the spine, at least one of the longitudinal member 12 and cross member 18 is aligned with the spine and the anatomical reference line, respectively. The alignment of the longitudinal member 12 with the spine and/or alignment of the cross member 18 with the anatomical reference line can then be compared to assure proper alignment of the spine during surgery.

The anatomical reference line of the method described above is preferably a reference line spanning a pelvis of a patient, a reference line spanning a first hip joint of a patient to a second hip joint of a patient, or a reference line spanning a first shoulder of a patient to a second shoulder of a patient. Thus, the anatomical intersection is the location where the selected anatomical reference line intersects the spine. The T-square device 50 of FIG. 5 would be advantageously used in the above described method to slide the cross member 52 along the longitudinal member 54 to align the cross member 52 with the selected anatomical reference line. The cross member 52 and longitudinal member 54 further include radiopaque marker/wires 56 to aid in measuring the anatomic dimensions of the spine and the selected anatomical reference.

There are many different features to the present invention and it is contemplated that these features may be used together or separately. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the disclosure set forth herein that are within scope and spirit of the present invention are to be included as further embodiments of the present invention. The scope of the present invention is accordingly defined as set forth in the appended claims.

What is claimed is:

1. A T-square medical device for intra-operative aligning of a spine with respect to anatomic references during spinal surgery, the T-square medical device comprising:
a cross member having a first visual marker, the first visual marker being configured to align with a first anatomical reference line; and
a longitudinal member connected orthogonally to the cross member, the longitudinal member having a second visual marker, the second visual marker being configured to align with a spinal reference line.

2. The T-square medical device according to claim 1, wherein the cross member is radiolucent.

3. The T-square medical device according to claim 1, wherein the cross member is radiopaque.

4. The T-square medical device according to claim 1, wherein the second visual marker is a radiopaque wire.

5. The T-square medical device according to claim 1, wherein a telescoping rod is disposed within the longitudinal member.

6. The T-square medical device according to claim 5, wherein the longitudinal member is extendable.

7. The T-square medical device according to claim 1, further comprising a further cross member having a third visual marker, the further cross member being connected orthogonally to the longitudinal member and parallel to the cross member.

8. The T-square medical device according to claim 7, wherein the third visual marker is configured to align with a second anatomical reference line.

9. The T-square medical device according to claim 8, wherein at least one of the first and second anatomical reference line is chosen from the group consisting of a reference line spanning a pelvis of a patient, a reference line spanning a first hip joint of a patient to a second hip joint of a patient, and a reference line spanning a first shoulder of a patient to a second shoulder of a patient.

10. A T-square medical device for intra-operative aligning of a spine with respect to anatomic references during fluoroscopic spinal surgery, the T-square medical device comprising:
a longitudinal member having a first radiopaque marker, the longitudinal member being configured to align to a spinal reference line; and
a cross member disposed orthogonal to the longitudinal member and having a second radiopaque marker, the cross member being configured to align to an anatomical reference line;
the cross member being operable to slide along the longitudinal member, and
the first and second radiopaque markers being operable to estimate anatomic dimensions during the fluoroscopic spinal surgery.

11. The T-square medical device according to claim 10, wherein the first radiopaque marker is a first group of radiopaque wires disposed along the longitudinal member and the second radiopaque marker is a second group of radiopaque wires disposed along the cross member.

12. The T-square medical device according to claim 10, wherein the cross member slides along the longitudinal member on a sliding dovetail connection.

13. The T-square device according to claim 12, wherein the cross member has a recess disposed below the cross member and the longitudinal member has a protrusion disposed above the longitudinal member, the protrusion engaging the recess to form the sliding dovetail connection.

14. The T-square medical device according to claim 10, wherein the cross member includes a first radiolucent marker configured to be positionable over a first femoral head of a patient and a second radiolucent marker configured to be positionable over a second femoral head of a patient.

15. The T-square medical device according to claim 10, wherein the anatomical reference line is selected from the group consisting of a reference line spanning a pelvis of a patient, a reference line spanning a first hip joint of a patient to a second hip joint of a patient, and a reference line spanning a first shoulder of a patient to a second shoulder of a patient.

16. A medical device for facilitating intra-operative alignment of a spine with respect to anatomic references during spinal surgery, the medical device comprising:
a first T-square part having a first longitudinal member and a first cross member, the first cross member being orthogonal to the first longitudinal member;
a second T-square part having a second longitudinal member and a second cross member, the second cross member being orthogonal to the second longitudinal member; and
means for joining the first and second longitudinal members;
the first cross member being configured to align to a first anatomical reference line, the second cross member being configured to align to a second anatomical reference line, and the joined first and second longitudinal members being a spinal reference line.

17. The medical device according to claim 16, wherein the first cross member is operable to slide along the first longitudinal member and the second cross member is operable to slide along the second longitudinal member.

18. The medical device according to claim 16, wherein the first T-square part and the second T-square part further comprises radiopaque markers.

19. The medical device according to claim 16, wherein the means for joining the first and second longitudinal member is a sliding dovetail connection.

20. The medical device according to claim 16, wherein at least one of the first and second anatomical reference line is chosen from the group consisting of a reference line spanning a pelvis of a patient, a reference line spanning a first hip joint of a patient to a second hip joint of a patient, and a reference line spanning a first shoulder of a patient to a second shoulder of a patient.

21. A method for aligning a spine with respect to an anatomical reference line, the anatomical reference line optimally being orthogonal to the spine and intersecting the spine at an anatomical intersection, the method which comprises:
mounting a longitudinal member orthogonally to a cross member, the longitudinal member and the cross member intersecting at a device intersection;
overlying the device intersection and the anatomical intersection;
aligning one of the longitudinal member and the cross member, with one of the spine and the anatomical reference line, respectively; and
comparing an alignment of the other of the longitudinal member with the spine and the cross member with the anatomical reference line.

22. The method for aligning a spine according to claim 21, further comprising:
selecting the anatomical reference line from the group consisting of a reference line spanning a pelvis of a patient, a reference line spanning a first hip joint of a patient to a second hip joint of a patient, and a reference line spanning a first shoulder of a patient to a second shoulder of a patient;
sliding the cross member along the longitudinal member to align the cross member with the selected anatomical reference line;
measuring anatomic dimensions of one of the spine or the selected anatomical reference line, with a radiopaque marker attached one of the longitudinal member or the cross member, respectively; and
aligning the spine after measuring the anatomic dimensions and comparing the alignment of the other of the longitudinal member with the spine and the cross member with the selected anatomical reference line.
